(19) United States
(12) Patent Application Publication
(10) Pub. No.: US 2011/0206813 A1
(43) Pub. Date: Aug. 25, 2011

(54) METHOD TO DETERMINE OXIDATIVE AND REDUCTIVE SUBSTANCES IN FOOD, TESTING SPECIMEN AND MEASURING DEVICE FOR THE SAME METHOD

(75) Inventors: Yen-Yuan SHEN, Taipei County (TW); Hsiu-Min Lin, Taipei County (TW); Edward Chen, Taipei County (TW)

(73) Assignee: EUMED BIOTECHNOLOGY CO., LTD.

(21) Appl. No.: 12/709,884

(22) Filed: Feb. 22, 2010

(51) Int. Cl. G01N 33/02; G01N 27/30

(52) U.S. Cl. 426/231; 204/405; 700/267

(57) ABSTRACT

A method to determine oxidative and reductive substances in food, which is realized with a testing specimen and a measuring device, and which comprises steps: pre-processing an appropriate amount of a food specimen; dissolving a given amount of the pre-processed food specimen in a buffer solution; making the buffer solution dissolving a tested material contact with a formulated layer on the testing specimen to proceed a redox reaction; the measuring device supplying a reaction potential to reaction electrodes of the testing specimen to obtain an electrical signal; and obtaining a content of the tested material from the electrical signal. Besides, the testing specimen and the measuring device used to implement the abovementioned method are also disclosed.

### FIELD OF THE INVENTION

The present invention relates to a method to test oxidative and reductive substances in food, particularly to a method to quantitatively test oxidative and reductive substances in food. The present invention also relates to a testing specimen and a measuring device to implement the abovementioned method.

### BACKGROUND OF THE INVENTION

Sulfur dioxide and sulfites are common food additives. Sulfur dioxide is a reductive bleaching having bleaching, decoloring, anti-oxidative, aseptic and disinfectant functions. Thus, sulfur dioxide is usually added to preserve foods via inhibiting the growth of microbes, such as saccharomycetes, mycetes and bacteria. Sulfites decompose into sulfur dioxide soon after being added to food. Thus, sulfites are also common food additives functioning like sulfur dioxide.

The sulfites that are usually added to food include sodium sulfite (Na₂SO₃), potassium sulfite (K2SO₃), sodium bisulfite (NaHSO₃), potassium bisulfite (KHSO₃), sodium metabisulfite (Na₂S₂O₅), potassium metabisulfite (K₂S₂O₅), and sodium hydrosulfite (Na₂S₂O₄). Sulfur dioxide or sulfites are usually added to the following foods: dried foods/foods for the lunar new year, including watermelon seeds, pistachio nuts, mushrooms, yellow daylily, bamboo fungus, and tremella fuciformis; fresh foods, including bamboo shoots, bean sprouts, meats, seafood; pickled/processed foods, including dried fruits, pickled cabbage, and candies. Sulfur dioxide or sulfites are also often used in disposable tableware and various wines. Long-term or overdose intake of sulfur dioxide will make a person feel uncomfortable and sick, affect the function of the stomach and intestines, impair the absorption of calcium and phosphorus, or even cause serious allergy or shock. Therefore, sulfur dioxide and sulfites may threaten the health of consumers.

According to the regulation of FAO (Food and Agriculture Organization of the United Nations) and WHO (World Health Organization), the allowed daily intake of sulfur dioxide or sulfite is about 0.7 mg/kg. Thus, the tolerance of sulfur dioxide for an adult weighing 60 kg is 42 mg per day. As to the content of sulfur dioxide/sulfite in food, every nation has its own standard. Therefore, it is meaningful and useful to quantitatively test the content of sulfur dioxide/sulfite.

Table.1 lists more than ten currently-available methods for testing the content of sulfur dioxide/sulfite. The content of sulfite is determined via quantitatively testing the content of the sulfur dioxide decomposed from the sulphite ions. Traditionally, qualitative tests can be realized by test paper. However, most of quantitative tests are undertaken in a laboratory by professional personnel with expensive equipment and complicated procedures.

An in-situ and fast quantitative test is hard to realize.

TABLE 1
comparison of methods for testing sulfur dioxide/sulfite in food

| Method | Advantage | Disadvantage |
|---|---|---|
| Acetate strip | In-situ and fast | Unable to quantify |
| | qualitative test method | sulfur dioxide, |
| | | only able to |
| | | perform |
| | | semi-quantification |
| | | of sulfur dioxide, |
| | | interfered by some |
| | | amino acids |
| Selective | Fast and low-cost, | Having to |
| electrode method | able to perform | appropriately |
| | continuous | select the reaction |
| | surveillance, usually | material lest the |
| | used to test sulfur | cost become too |
| | dioxide in gas | high |
| Rankine method | Legal test method for | Complicated |
| | sulfur dioxide in food | equipment, |
| | (in Taiwan), accurate | time-consuming |
| | and high | operation process, |
| | reproducibility | likely to be |
| | | influenced by |
| | | specimen size, |
| | | sample amount, |
| | | heating time, |
| | | phosphoric acid |
| | | concentration, |
| | | needing a nitrogen |
| | | cylinder to supply |
| | | nitrogen, unlikely |
| | | to perform in situ |
| Modified | High reproducibility | Complicated |
| Monier-Williams | | equipment, |
| method | | time-consuming |
| | | operation process |
| | | (2 hours for each |
| | | specimen) |
| Sulfite oxidative | Applicable to | Applying to |
| enzyme method | electrochemical | colormetry, |
| | analysis | complicated |
| | | analysis process |
| Direct titration | Extensively used to | The reproducibility |
| method | perform an in-situ and | thereof inferior to |
| | fast quantitative test of | the modified |
| | sulfur dioxide, and | Monier-Williams |
| | using simple | method, likely to |
| | apparatuses, shorter | be interfered by |
| | test time, available in | reductive |
| | common laboratory | substances, such as |
| | | formaldehyde and |
| | | hydrogen peroxide |
| Micro-diffusion | Using a small airtight | Unable to perform |
| mehtod | culture dish, sulfur | a quantitative test |
| | dioxide diffusing, | |
| | reacting and | |
| | pigmenting in the | |
| | culture dish, able to | |
| | perform in situ | |
| Distillation-pigmentation | | Needing a heating |
| method | | device, using |
| | | complicated |
| | | procedures, unable |
| | | to perform in situ |
| Pararosaniline | The most sensitive and | Needing a |
| hydrochloride | accurate method, | spectrometer to |
| color test | suitable to test various | perform |
| | concentrations of | colorimetric |
| | sulfur dioxide | analysis at a |
| | | wavelength of |
| | | 552 nm |
| Flow injection | Accurate | Performed in a |
| analysis | | laboratory with |
| | | large-scale and |
| | | expensive |
| | | equipment |
| Ion exchange | Accurate | Performed in a |
| chromatography | | laboratory with |
| | | large-scale and |
| | | expensive |
| | | equipment |
| High performance | Accurate | Performed in a |
| liquid | | laboratory with |
| chromatography | | large-scale and |
| | | expensive |
| | | equipment |
| Gas | Accurate | Performed in a |
| chromatography | | laboratory with |
| | | large-scale and |
| | | expensive |
| | | equipment |
| Differential pulse | Accurate | Performed in a |
| polarographic | | laboratory with |
| method | | large-scale and |
| | | expensive |
| | | equipment |

### SUMMARY OF THE INVENTION

The primary objective of the present invention is to provide a simple method to determine oxidative and reductive substances in food, a testing specimen and a measuring device to implement the method, whereby the determination can be performed in situ or in a mobile way, and whereby is avoided the expensive in-laboratory test.

Another objective of the present invention is to provide a simple method to determine oxidative and reductive substances in food, a testing specimen and a measuring device to implement the method, whereby the user or consumer can easy and fast learn whether the content of a specified oxidative or reductive material exceeds the standard of food additives, and whereby the user or consumer can prevent from taking foods harmful to health.

To achieve the abovementioned objectives, the present invention proposes a method to determine oxidative and reductive substances in food, which is implemented with a testing specimen and a measuring device, and which comprises steps: pre-processing an appropriate amount of the food specimen; dissolving a given amount of the pre-processed food specimen in a buffer solution; making the buffer solution dissolving the tested material reacts with a formulated layer of a testing specimen to proceed a redox reaction; a measuring device supplying a reaction potential to a reaction electrode of the testing specimen to obtain an electrical signal; and obtaining the content of the tested material from the electrical signal.

In the present invention, the composition of the formulated layer of the testing specimen varies with the different tested material to test different oxidative/reductive material in food. The measuring device receives an electrical signal from the testing specimen and analyzes the electrical signal to obtain the concentration of the tested material.

Via the present invention, the user can learn the concentration of the oxidative/reductive substances in food in a very short time. Thus, unqualified foods can be removed, and food safety of the people is protected.

Below, the embodiments are described in detail in cooperation with the drawings to demonstrate the technical contents of the present invention.

### BRIEF DESCRIPTION OF THE DRAWINGS

The embodiments of the present invention are to be described in cooperation with the following drawings:

FIG. 1A is a diagram schematically showing the electron transfer in a redox reaction on the working electrode according to the present invention;

FIG. 1B is a diagram showing the relationship of potential and time and the relationship of current and time in the potential step voltammetry of electrochemistry;

FIG. 2A and FIG. 2B are diagrams showing the calibration lines obtained with the potential step voltammetry according to the present invention;

FIG. 3 is a flowchart of a method to test oxidative and reductive substances in food according to the present invention;

FIG. 4 is a perspective view schematically showing the appearance of a testing specimen according to one embodiment of the present invention;

FIG. 5 is an exploded view schematically showing a testing specimen according to one embodiment of the present invention;

FIG. 6 is a perspective view schematically showing the appearance of a measuring device according to one embodiment of the present invention; and

FIG. 7 is a block diagram schematically showing the architecture of a measuring device according to one embodiment of the present invention.

### DETAILED DESCRIPTION OF THE PREFERRED EMBODIMENTS

The method of the present invention cooperates with a testing specimen and a measuring device to qualitatively and quantitatively analyze oxidative and reductive substances in food. The method of the present invention can determine the natural oxidative/reductive substances, the natural/synthetic oxidative/reductive additives, and the derivatives thereof. When using an oxidative mediator, the method of the present invention can determine reductive substances in food, such as sulfur dioxide, sulfites, vitamin C, formaldehyde, Rongalite, and phenol. When using a reductive mediator, the method of the present invention can determine oxidative substances in food, such as hydrogen peroxide, manganese oxide, chromium oxide, chlorine and chlorine oxide. Sulfur dioxide and sulfites are the primary food additives for bleaching or reducing. Therefore, the present invention will be exemplified with the applications to sulfur dioxide and sulfites. The test method, testing specimen and measuring device of the present invention for other oxidative and reductive substances are similar to those for sulfur dioxide and sulfites. Therefore, only the difference thereof will be further described in addition to the test method, testing specimen and measuring device for sulfur dioxide and sulfites. Since similarity exists in various embodiments of the present invention, some omitted technical details of the applications to other oxidative and reductive substances should not influence the readers' understanding to the present invention.

Below, the test method, testing specimen and measuring of the present invention are respectively described in three sections.

### The Test Method

The principle of the test method of the present invention will be demonstrated with the application to sulfur dioxide. Sulfur dioxide (SO₂) dissolving in water or a basic solution will form sulfite ions (SO₃²⁻). Therefore, sulfur dioxide and sulfite are substances able to mutually convert into each other. Sulfur dioxide (SO₂)/sulfite (SO₃²⁻) is a reductive agent able to react with some ionic oxidative agent in water to form sulfuric ions (SO₄²⁻); the ionic oxidative agent is reduced into a reductive ion. The abovementioned electrochemical reaction can be used to determine sulfur dioxide and sulfites and can be expressed by Formula (1):

SO₃²⁻+ionic oxidative agent+H₂O→SO₄²⁻+reductive ion+2H⁺ (1)

The ionic oxidative agent in Formula (1) is a redox mediator usually used in an electrochemical reaction and simultaneously appearing in form of an oxidative ion and a reductive ion in the same solution. According to the potential step voltammetry of electrochemistry, a given electrical potential is applied to the reaction electrodes to oxidize the reductive ion in Formula (1), whereby the reductive ion is converted back to the oxidative ion and electrons are released, as shown in Formula (2):

reductive ions→oxidative ions+e⁻ (2)

At the same time, the reaction electrodes conduct the electrons, and thus the corresponding electrical signal varying with time can be detected. The variation of the electrical signal correlates with the concentration of the reductive ion and the ratio of reductive ion and oxidative ion in Formula (1). Thus, the content of sulfur dioxide or sulfite can be quantitatively analyzed according to the abovementioned principle.

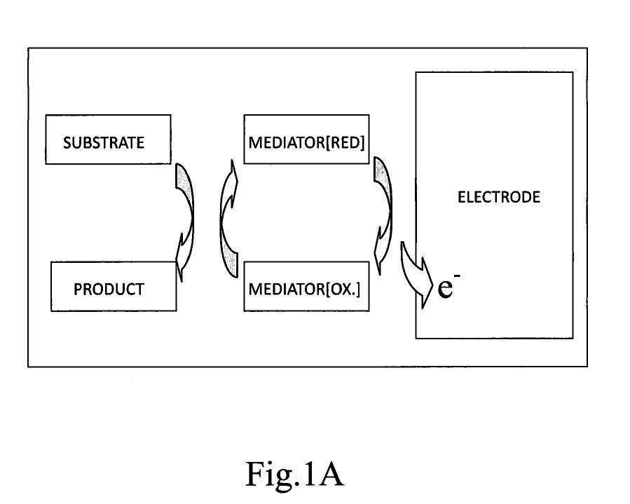

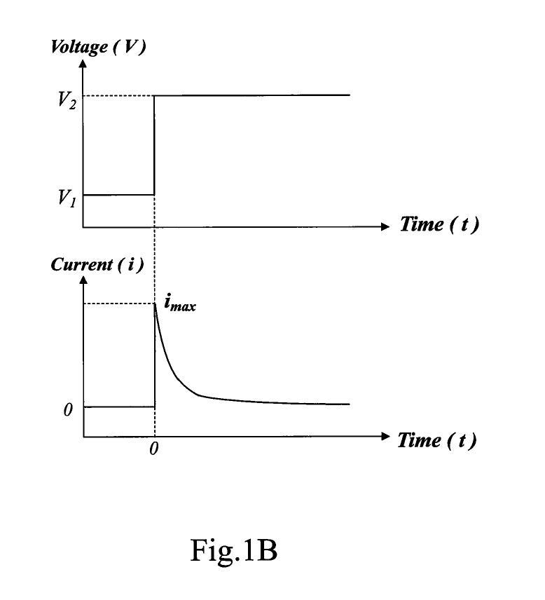

Refer to FIG. 1A a diagram schematically showing the abovementioned principle. Suppose that the tested substrate is a reductive redox material. An oxidative mediator is placed on the electrodes and reacts with the reductive substrate. Thus, the oxidative mediator is partially converted into a reductive mediator, and the electrodes contact the mixture of the oxidative mediator and reductive mediator.

Refer to FIG. 1B. Initially, an electrical potential V₁ is applied to the electrodes. At the instant, neither a redox reaction appears in the mediator nor occurs a current in the electrodes. Then, the electrical potential is quickly increased to V₂, and the oxidization reaction of the reductive mediator performs at the maximum rate and generates the maximum current iₘₐₓ with the reduction reaction completely inhibited. Thus, the reductive mediator on the surface of the electrodes is consumed, and the concentration thereof decreases, with the current generated thereby decreasing. Then, the oxidization reaction is maintained by the diffusion of the reductive mediator from the solution to the surface of the electrodes, and the current decreases and finally reaches an equilibrium value. According to Formula (3)—the Cottrell equation, the Faraday current generated in the electrodes is proportional to the concentration of the reductive mediator converted from the substrate. At a given time point after applying the electric potential, the currents can be measured via applying an electrical potential to the reductive mediator converted from different concentrations of the substrate. Then, a relationship between the currents and the concentrations of the substrate can be plotted. Thus is obtained a straight line having a slope of (nFAD^{1/2}/π^{1/2}) according to Formula (3):

$$\vert i \vert = \frac{{nFA [ Reactant ]}_{Bulk} \sqrt{D}}{\sqrt{π} \times \sqrt{t}} \qquad ( 3 )$$

wherein n is the number of the electrons transferred in the electrochemical reaction, F the Faraday constant, A the area of the electrode, D the diffusivity of the reactant, [Reactant]_{bulk} the concentration of the reactant.

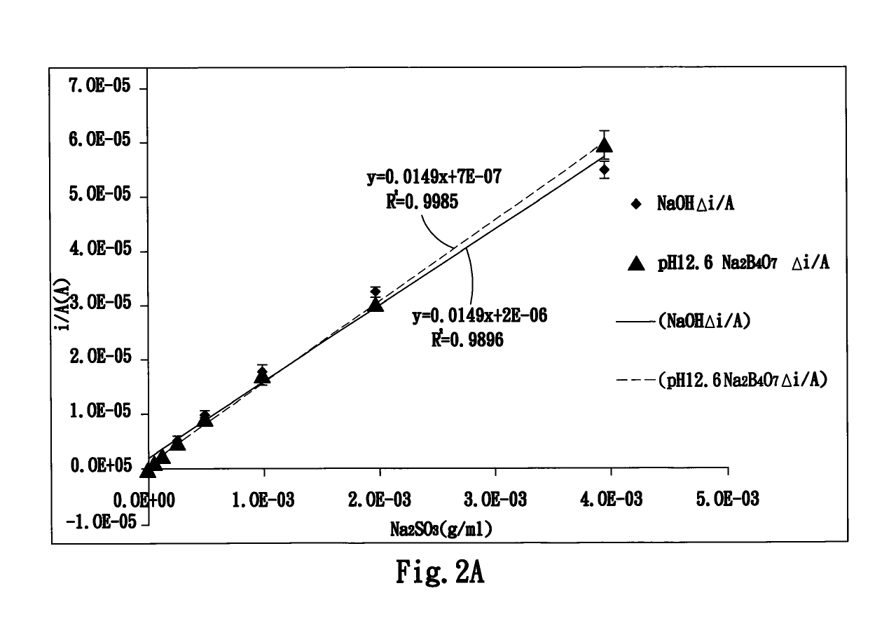

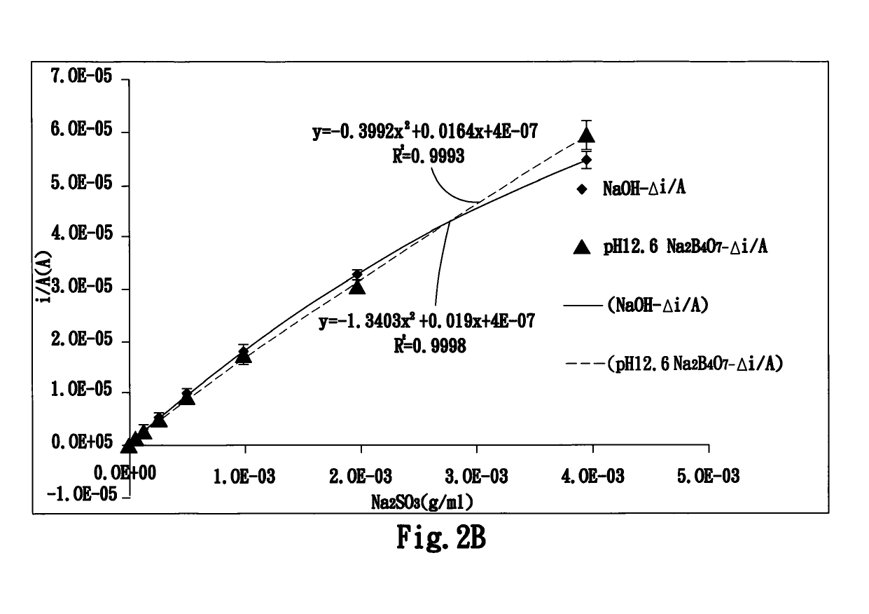

Refer to FIG. 2A and FIG. 2B for the experimentally-obtained relationships of the currents and the concentrations of the substrate, wherein the vertical axis denotes the current and the horizontal axis denotes the concentration of the substrate. FIG. 2A and FIG. 2B show that the experimental results are very close to the theoretical linearity relationship. To achieve a more precise result, the data obtained in several experiments are further analyzed with a first-order or second-order linear regression to attain a calibration line.

As shown in FIG. 1B, an equilibrium current can be determined after the external electrical potential has been applied for a given interval of time. According to the linearity relationship (the calibration line) in FIG. 2, the corresponding concentration of the substrate can be learned from the determined current. According to the Ohm's law, a relationship exists among current, voltage and resistance. Thus, the concentration of a tested material can also be obtained via determining resistance or voltage.

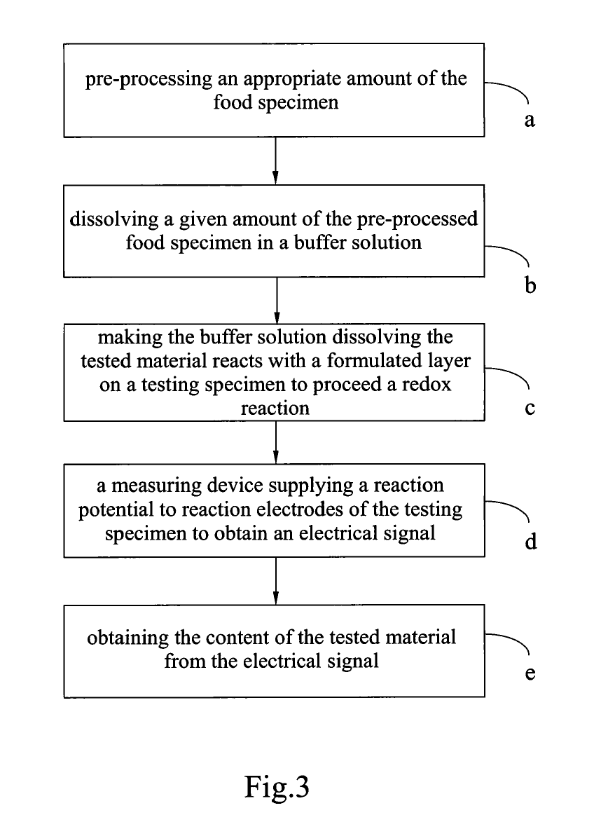

According to the abovementioned principle, the present invention proposes a method for determining oxidative and reductive substances in food. Refer to FIG. 3. The method of the present invention comprises steps:
a. pre-processing an appropriate amount of the food specimen;
b. dissolving a given amount of the pre-processed food specimen in a buffer solution;
c. making the buffer solution dissolving the tested material reacts with a formulated layer on a testing specimen to proceed a redox reaction;
d. a measuring device supplying a reaction potential to reaction electrodes of the testing specimen to obtain an electrical signal; and
e. obtaining the content of the tested material from the electrical signal.

Below, the steps are respectively described in detail.

The present invention does not intend to differentiate food from food products by whether they are processed. In the present invention, both food and food products are referred to the eatables for human beings. The pre-processing in Step a is to make a food specimen suitable for test, such removing impurities or granulation. Granulation makes the tested material (such as sulfur dioxide or sulfite) in food easy to dissolve in the buffer solution in Step b. Granulation may be realized via cutting, grinding, crushing, agitating, tweaking, or tearing. The present invention does not limit the method of granulating. However, the user has to consider the factor: some granulation methods, such as grinding, may accelerate the dissipation or volatilization of the tested material and increase the area and probability of oxidization, and are thus likely to increase the determination errors. Step a is to process the food into chips or crumbs, wherein the tested material of the food is hard to dissolve. Step a is unnecessary for powdered or liquid food. Herein, granulation does not literally refer to grinding food into small pieces or grains but only refer to reducing the volume of food.

In Step b, the given amount of the pre-processed food specimen is placed in the buffer solution to dissolve the tested material into the buffer solution. For example, place 1 g of the food specimen in 1 or 2 ml of the buffer solution for several minutes. In one embodiment, the buffer solution has a pH value greater than 3 and has concentration of 0.05-2M, wherein the buffer solution can be selected from citrate/citric acid, phosphate/phosphoric acid, carbonate/carbonic acid, borax/boric acid, or a base of low concentration. In another embodiment, the buffer solution is pure water. For example, if the tested material is sulfur dioxide or sulfite, the buffer solution may be a sodium hydroxide solution (e.g. 0.15N), a borax (sodium borate) solution (e.g. pH9-13, 0.1M), or water. The buffer solution should not react with the tested material or the formulated layer of the testing specimen (described in detail later). Thus, the buffer solution needs selecting according to the tested material. The reason why the pre-processed food specimen is taken by the given amount is to fix the dilution ratio and the concentration of the food specimen. In one embodiment, after the food specimen is placed in the buffer solution, the food specimen and buffer solution is stirred, vibrated, swung or shaken to accelerate the dissolution of the tested material. Herein, it should be mentioned: for some liquid food, Step b can be omitted, and an appropriate amount of the liquid food is taken to directly react with a reaction region of the testing specimen.

In Step c, the buffer solution dissolving or suspending the tested material contacts with the formulated layer of the testing specimen to perform the redox reaction. In one embodiment, the buffer solution of 0.5-1000 μl dissolving the tested material is taken to react with the formulated layer of the testing specimen. In another embodiment, the buffer solution is sucked to the reaction region of the testing specimen via a capillary or siphon method. Step c is to make the buffer solution dissolving the tested material react with a testing specimen. The structure of the testing specimen is to be described in detail later.

In Step d, the measuring device provides the reaction potential to the reaction electrodes of the testing specimen to obtain the electrical signal. The reaction electrodes include a working electrode and a counter electrode, which will be described in detail later. The electrical signal may be the value of current, resistance or voltage. In an example that the electrical signal is the value of current, the testing specimen is inserted into the measuring device firstly; the testing specimen reacts with the buffer solution dissolving the tested material, whereby is triggered the measuring device to count for 1-60 seconds. After 1-60 seconds of reaction time, the measuring device supplies the reaction potential (e.g. 0.01-1.5V) to one reaction electrode of the testing specimen and obtains a responsive electrical signal (a value of current, voltage or resistance), as mentioned in the description of the reaction principle.

In Step e, the concentration of the tested material in food is deduced from the value of the electrical signal according to the calibration line. The measuring device further has a display unit from which the user can read the concentration of the tested material.

The method of the present invention can determine the natural/synthetic oxidative/reductive substances, and the derivatives thereof. The reductive substances the present invention can test include sulfur dioxide, sulfites, vitamin C, formaldehyde, Rongalite and phenol. The oxidative substances the present invention can determine include hydrogen peroxide, manganese oxide (MnO₂, KMnO₄), chromium oxide (CrO₃, K₂Cr₂O₇), chlorine (Cl₂), chlorine oxide and ozone (O₃). In one embodiment for testing sulfur dioxide, the detectable concentration of SO₂ ranges from 0 to 4000 mg/L. In one embodiment for testing sulfites, the detectable concentration of Na₂SO₃ ranges from 60 to 7800 mg/L. The zero concentration indicates that none tested material exists in the food specimen or that the concentration of the tested material is lower than the detection limit of the measuring device.

Table.2 and Table.3 respectively show the buffer solutions and the substances of the reaction electrodes of the testing specimens for determining reductive substances and oxidative substances.

TABLE 2

| | Oxidative | | Applied |
|---|---|---|---|
| Tested material | mediator | Buffer solution | potential |
| aqueous solution | potassium | NaOH, Na₂B₄O₇, | 0.3 V~0.6 V |
| of sulfur dioxide | ferricyanide | H₂O | (working |
| | | | electrode) |
| aqueous solution | potassium | NaOH, Na₂B₄O₇, | 0.3 V~0.6 V |
| of sulfite | ferricyanide | H₂O | |
| aqueous solution | potassium | NaOH, Na₂B₄O₇, | 0.3 V~0.6 V |
| of vitamin C | ferricyanide | H₂O | |
| aqueous solution | potassium | NaOH, Na₂B₄O₇, | 0.3 V~0.6 V |
| of formaldehyde | ferricyanide | H₂O | |
| aqueous solution | potassium | NaOH, Na₂B₄O₇, | 0.3 V~0.6 V |
| of Rongalite | ferricyanide | H₂O | |
| (sodium | | | |
| sulphoxylate | | | |
| formaldehyde) | | | |
| aqueous solution | potassium | NaOH, Na₂B₄O₇, | 0.3 V~0.6 V |
| of phenol or | ferricyanide | H₂O | |
| polyphenol | | | |

TABLE 3

| | Reductive | | Applied |
|---|---|---|---|
| Tested material | mediator | Buffer solution | potential |
| aqueous solution | potassium | HCl, H₂O | −0.3 V~−0.6 V |
| of manganese | ferrocyanide | Citrate, | (working |
| oxise | | Acetate acid | electrode, |
| (MnO₂, KMnO₄) | | | providing |
| | | | electrons for |
| | | | oxidized |
| | | | mediator) |
| aqueous solution | potassium | HCl, H₂O | −0.3 V~−0.6 V |
| of chromium oxide | ferrocyanide | Citrate, | (working |
| (CrO₃,K₂Cr₂O₇) | | Acetate acid | electrode) |
| aqueous solution | potassium | HCl, H₂O | −0.3 V~−0.6 V |
| of chlorine | ferrocyanide | Citrate, | (working |
| (Cl₂) | | Acetate acid | electrode) |
| aqueous solution | potassium | HCl, H₂O | −0.3 V~−0.6 V |
| of chlorine oxide | ferrocyanide | Citrate, | (working |
| (ClO, ClO₄) | | Acetate acid | electrode) |
| aqueous solution | potassium | HCl, H₂O | −0.3 V~−0.6 V |
| of ozone | ferrocyanide | Citrate, | (working |
| (O₃) | | Acetate acid | electrode) |

### The Testing Specimen

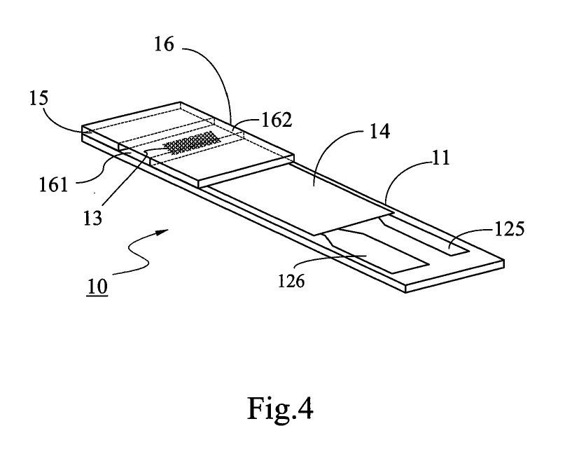

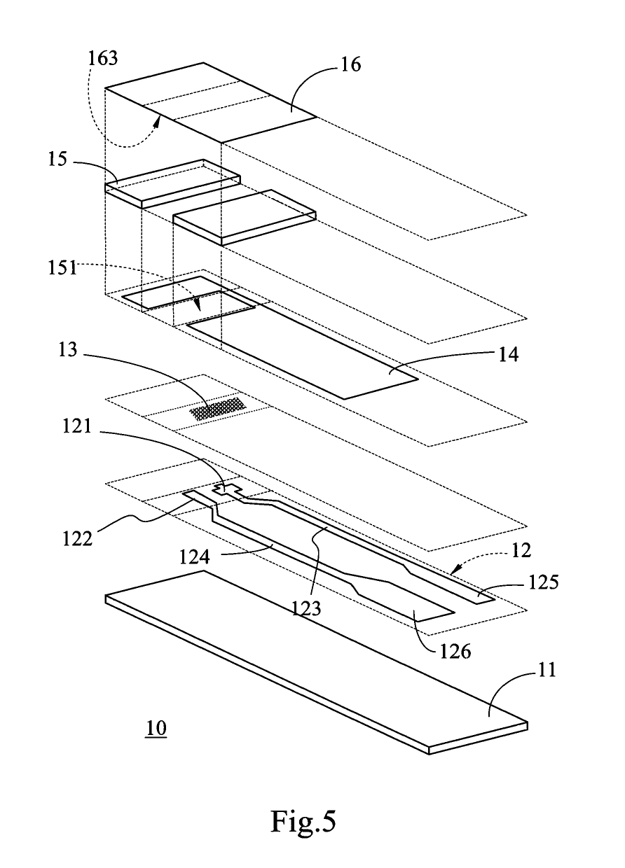

Refer to FIG. 4 and FIG. 5 respectively a perspective view and an exploded view of a testing specimen according to one embodiment of the present invention. The testing specimen 10 of the present invention has a working electrode 121, a counter electrode 122 and a formulated layer 13. Two conductive wires 123 and 124 respectively connect with the working electrode 121 and the counter electrode 122 to form connection terminals 125 and 126 on one end of the testing specimen 10. The connection terminals 125 and 126 can electrically connect with a measuring device, whereby the measuring device can receive an electrical signal from the testing specimen 10 to quantitatively determine the content of the tested material. The formulated layer 13 is arranged above the working electrode 121 and the counter electrode 122. The formulated layer 13 has different chemical formulae to react with the buffer solutions dissolving different tested materials.

As shown in FIG. 5, the testing specimen 10 of this embodiment comprises a substrate 11, a working layer 12, the formulated layer 13, an insulation layer 14, a spacer layer 15 and a top layer 16. The substrate 11 is the support material of the testing specimen 10. The electrodes are formed on the substrate 11 to form the working layer 12 with an appropriate method, such as a transfer-printing method, an etching method, a vapor deposition method, a sputtering method, a screen-printing method, a relief-printing method, or a marking press method. The working layer 12 includes the working electrode 121, the counter electrode 122, the conductive wires 123 and 124, the connection terminals 125 and 126. The formulated layer 13 is arranged above the working electrode 121 and the counter electrode 122.

The substrate 11 is flat and hard to thermally deform. The substrate 11 has sufficient structural strength and superior electrically-insulation ability. The substrate 11 is made of a plastic material, a glass material, a glass fiber material, or a ceramic material. The plastic substances for the substrate 11 include polyvinylchloride (PVC), polyester, polyethylene terephthalate (PET), polycarbonate (PC), polypropylene (PP), polyethylene (PE), polybutylene (PBT), polystyrene (PS), polyethylene polyvinylidene fluoride (PVDF), polyamide (PA), and Bakelite.

The material of the working layer 12 may adopt the conventional material of the electrode and conductive wire used in an electrochemical test, including conductive carbon and metals, such as gold, silver, palladium, platinum and silver chloride. In one embodiment, the conductive carbon is printed on the substrate 11 with the screen-printing method. An auxiliary conductive layer (not shown in the drawing) is formed below the working layer 12 to reduce the resistance of the carbon material. The auxiliary conductive layer is made of a material having a higher conductivity, such as silver, copper or tin. If the working layer 12 is formed with the vapor deposition method or the sputtering method, a metallic adhesion layer (not shown in the drawing) may be formed between the substrate 11 and the working layer 12 to assist the working layer 12 in adhering to the substrate 11. The metallic adhesion layer may be made of chromium or nickel.

The present invention does not particularly limit the shape of the working electrode 121 and the counter electrode 122. The working electrode 121 or the counter electrode 122 may have a shape selected from a group consisting of a square, a rhombus, a trapezoid, a rectangle, a circle, an ellipse, an asterisk, a heart, and other polygons.

The formulated layer 13 is the region to react with the buffer solution dissolving the tested material. The formulated layer 13 has different formulae corresponding to the different tested substances. A formulated liquid is placed on the working electrode 121 and the counter electrode 122 and then dried to form the formulated layer 13. The formulated liquid is placed on the working electrode 121 and the counter electrode 122 with a smearing method, a printing method, a spraying method, or a dripping method. The formulated layer 13 may be a mixture included a redox agent, a mediator, a buffer salt, or a surfactant. The formula can detect the oxidative/reductive substances in food. Refer to Table.2 and Table.3 for the tested substances and the formulae of the corresponding formulated layers 13. In one embodiment, the mediator may be potassium ferricyanide, potassium ferrocyanide, p-benzoquinone, phenazine methosulfate, ferrocene, indophenols, or methylene blue.

In another embodiment, a bioactive material is added to the formulated layer 13 to form a biological sensor. The bioactive material is selected from a group consisting of glucose oxidase, glucose dehydrogenase, lactate oxidase, lactate dehydrogenase, fructose oxidase, fructose dehydrogenase, galactose oxidase, cholesterol oxidase, cholesterol dehydrogenase, cholesterol esterase, alcohol oxidase, alcohol dehydrogenase, and the derivatives thereof.

The insulation layer 14 covers the working layer 12 but exposes the formulated layer 13, the connection terminals 125 and 126, the working electrode 121 and the counter electrode 122. The spacer layer 15 overlays the insulation layer 14 and exposes the formulated layer 13 to form a reaction region 151. The connection terminals 125 and 126 and the working electrode 121 and counter electrode 122 are also exposed by the spacer layer 15. The top layer 16 is arranged over the reaction region 151 to form a two-opening channel. One opening is a specimen inlet 161 where the buffer solution dissolving the tested material enters the reaction region 151 via a capillary method or a siphon method. The other opening is an air outlet 162, and the flow of the specimen also ends at the air outlet 162.

In one embodiment, a hydrophilic layer is formed on an inner surface 163 of the top layer 16, which faces the reaction region 151. The hydrophilic layer is formed via modifying the inner surface 163 with a hydrophilic molecule, such as sulfonyl, sulfonate or carboxyl. Alternately, the hydrophilic layer may be formed via coating or applying a hydrophilic material onto the inner surface 163. The hydrophilic material is selected from a group consisting of ethyl cellulose, methyl cellulose, hydroxypropyl cellulose, cellulose acetate, nitrocellulose, polyvinyl pyrrolidone, polysulfone, polyvinylidene fluoride, polyamide and polyimide.

### The Measuring Device

The present invention proposes a portable measuring device 20, which applies a reaction potential to the testing specimen 10 and obtains a responsive electrical signal. The measuring device 20 analyzes the electrical signal to obtain the concentration of the tested material.

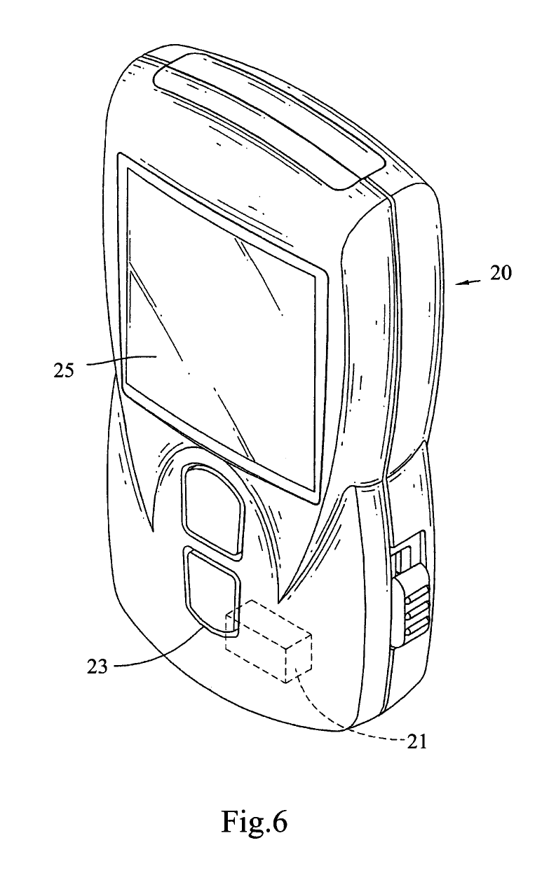

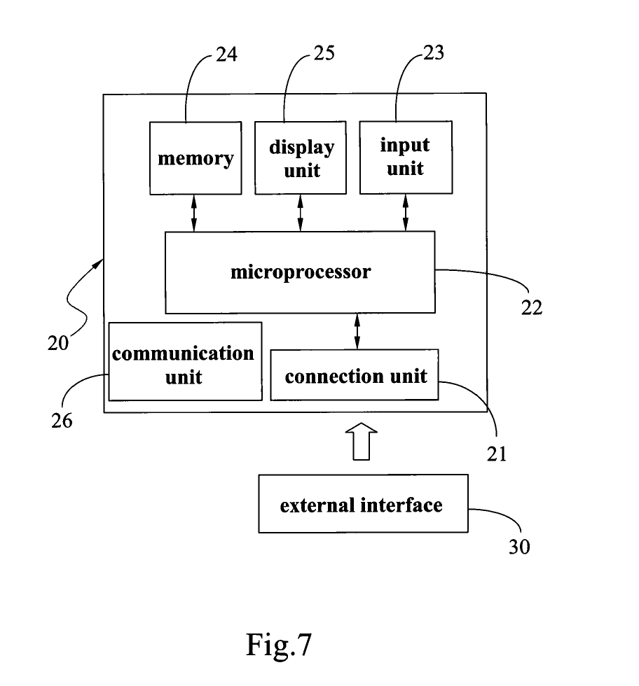

Refer to FIG. 6 and FIG. 7. FIG. 6 is a perspective view schematically showing the appearance of a measuring device according to one embodiment of the present invention. FIG. 7 is a block diagram of the architecture of a measuring device according to one embodiment of the present invention. The measuring device 20 comprises a connection unit 21, a microprocessor 22, an input unit 23, a memory 24 and a display unit 25.

The connection unit 21 has a plurality of pins for connecting with an external interface 30. The structure, spacing and size of the pins are designed to enable the different external interfaces 30 to plug in the connection unit 21, as shown by the arrow in FIG. 7. The external interfaces 30 include the testing specimen 10. The connection terminals 125 and 126 of the testing specimen 10 are inserted into the connection unit 21 to perform a determination. The external interfaces 30 also include a calibration specimen for calibrating the measuring device 20 and parameter specimens providing test parameters of the different tested substances.

The microprocessor 22 undertakes control, tests, calibration, data transmission, data storage, etc. The microprocessor 22 uses an interface identification process to identify an external interface 30 and then executes the procedures corresponding to the external interface 30. The microprocessor 22 includes amplifiers, digital-analog converters, and other necessary elements, but they will not be further described herein.

The input unit 23 is a press button device. The user operates the input unit 23 to adjust or control the functions of the measuring device 20. The user can use the input unit 23 to perform related setting, such timing, the unit for measurement, reading or deleting records, etc. The memory 24 connects with the microprocessor 22. The memory 24 stores the test data and the data read from the external interface 30. The memory 24 may be integrated with the microprocessor 22. The display unit 25 presents the operation status and the test results.

In one embodiment, the measuring device 20 further comprises a communication unit 26 linking to an external information device for exchanging information and controlling the measuring device 20. The communication unit 26 has a USB interface, an RS-232 interface or another communication port to link to an external information device, such as a computer or a mobile phone, for exchanging information and controlling the measuring device 20. In one embodiment, the measuring device is powered by an external power source or a built-in battery (not shown in the drawing). In one embodiment, the measuring device 20 further comprises a transmission unit (not shown in the drawing) able to link to a computer's transmission module (such as a USB connector). In one embodiment, the measuring device 20 can detect an analog current of 0-250 μA. In one embodiment, the measuring device 20 can work at a temperature of 0-60° C. and has a temperature compensation function. In one embodiment, the measuring device 20 can present the concentration of the tested material in different measurement units, such as mg/Kg, mg/L and ppm.

When the testing specimen 10 is inserted into the connection unit 21, the microprocessor 22 immediately sends a potential signal to specified pins of the testing specimen 10 and detects the current response from the pins. As the electrodes of the testing specimen 10 is in an open-circuit state, the potential signal will have a very weak responsive current. According to the responsive current and the built-in look-out table, the microprocessor 22 determines that the inserted interface is the testing specimen 10 and then enters into the test mode. According to the principle described above, different concentrations of the tested material will generate different reaction currents. The detected analog electrical signal can be converted into the concentration of the tested material according to the calibration line. In other words, the detected current is substituted into the conversion equation read from the parameter piece or built in the microprocessor 22 to obtain the concentration of the tested material.

Different tested substances respectively have different relationships of concentration and current. The present invention applies to many tested substances. Thus are established different calibration lines for different tested substances in the present invention. In one embodiment, the data of the calibration line is stored in the parameter piece, and the measuring device 20 reads the data from the parameter piece before testing. In one embodiment, the calibration lines are built in the measuring device 20, and the user uses the input unit 23 to select an appropriate calibration line before testing.

The measuring device 20 of the present invention is designed to have a handheld size to realize portability, whereby the user can carry about the measuring device 20 in his pocket. Further, the testing specimens 10, the measuring device 20 and a small amount of buffer solution may be packaged into a portable test kit or a desktop measuring device to realize an in-situ test or a dynamic test.

The embodiments described above are only to exemplify the present invention but not to limit the scope of the present invention. Any equivalent modification or variation according to technical contents of the specification or the drawings is to be also included within the scope of the present invention.

What is claimed is:

1. A method to determine oxidative and reductive substances in food, comprising steps:
pre-processing an appropriate amount of a food specimen;
dissolving a given amount of the pre-processed food specimen in a buffer solution;
making the buffer solution dissolving a tested material reacts with a formulated layer on a testing specimen to proceed a redox reaction;
a measuring device supplying a reaction potential to a reaction electrode of the testing specimen to obtain an electrical signal; and
obtaining a content of the tested material from the electrical signal.

2. The method to determine oxidative and reductive substances in food according to claim 1, wherein the tested material is sulfur dioxide or sulfite.

3. The method to determine oxidative and reductive substances in food according to claim 2, which is able to detect sulfur dioxide having a concentration of 0-4000 mg/L and sulfite having a concentration of 0-7800 mg/L.

4. The method to determine oxidative and reductive substances in food according to claim 1, wherein the pre-processing further comprises a granulation step to increase surface area of the food specimen and make the tested material in the food specimen dissolve in the buffer solution easily.

5. The method to determine oxidative and reductive substances in food according to claim 1, wherein the buffer solution is selected a group consisting of boric acid, borate, carbonic acid, carbonate, phosphoric acid, phosphate, citric acid, citrate, a base of low concentration and water.

6. The method to test oxidative and reductive substances in food according to claim 1, wherein 0.5-1000 μL of the buffer solution dissolving the tested material is taken to react with the formulated layer of the testing specimen.

7. The method to determine oxidative and reductive substances in food according to claim 1, wherein the buffer solution dissolving the tested material is sucked to the testing specimen via a capillary method or a siphon method.

8. A testing specimen applying to the method to determine oxidative and reductive substances in food according to claim 1, comprising a working layer and a formulated layer, and the working layer further comprises a working electrode and a counter electrode, and the formulated layer is arranged over the working electrode and the counter electrode, and wherein two conductive wires respectively connect with the working electrode and the counter electrode and form connection terminals on one end of the testing specimen, and wherein the connection terminals can electrically connect with the measuring device, whereby the measuring device can detect the content of the tested material.

9. The testing specimen according to claim 8, wherein the formulated layer further comprises a redox agent, a mediator, a buffer salt and a surfactant, and wherein the mediator is a material selected from a group consisting of potassium ferricyanide, potassium ferrocyanide, p-benzoquinone, phenazine methosulfate, ferrocene, indophenols, and methylene blue.

10. The testing specimen according to claim 8 further comprising a substrate, an insulation layer, a spacer layer and a top layer, wherein the working layer is arranged over the substrate, and wherein the insulation layer covers the working layer but exposes the formulated layer, the connection terminals, the working electrode and the counter electrode, and wherein the spacer layer is arranged over the insulation layer and exposes the formulated layer to form a reaction region, and wherein the top layer is arranged over spacer layer to form a two-opening channel above the reaction region.

11. The testing specimen according to claim 10, wherein the substrate is made of a material selected from a group consisting of polyvinylchloride, polyester, polyethylene terephthalate, polycarbonate, polypropylene, polyethylene, polybutylene, polystyrene, polyethylene polyvinylidene fluoride, polyamide, Bakelite, a glass material, a glass fiber material, and a ceramic material.

12. The testing specimen according to claim 10, wherein a hydrophilic layer is formed on an inner surface of the top layer, which faces the reaction region.

13. A measuring device applying to the method to determine oxidative and reductive substances in food according to claim 1, comprising
a connection unit having at least one pin for connecting with an external interface, wherein the specification of the pins are corresponding to the external interface to plug in the connection unit;
a microprocessor using an interface identification process to automatically identify the external interface and then executes procedures corresponding to the external interface;
an input unit, wherein the user operates the input unit to adjust or control the functions of the measuring device;
a memory connecting with the microprocessor and storing test-related data; and
a display unit presenting an operation status and test results of the measuring device.

14. The measuring device according to claim 13 further comprising a built-in battery supplying power to the measuring device, and the measuring device can be fabricated into a portable measuring device or a desktop measuring device.

15. The measuring device according to claim 13 storing a plurality of calibration lines for testing different tested materials, wherein the user uses the input unit to select an appropriate calibration line.

16. The measuring device according to claim 13, wherein the external interface is the testing specimen.

17. The measuring device according to claim 13 further comprising a communication unit linking to an external information device for exchanging information and controlling the measuring device, wherein the communication unit has a USB interface or an RS-232 interface.

18. The measuring device according to claim 13, wherein the testing specimens, the measuring device and a small amount of the buffer solution are packaged into a portable test kit.

19. The measuring device according to claim 13 able to detect an analog current of 0-250 μA.

20. The measuring device according to claim 13 working at a temperature of 0-60° C. and having a temperature compensation function
